(19) United States
(12) Patent Application Publication
(10) Pub. No.: US 2005/0148658 A1
(43) Pub. Date: Jul. 7, 2005

(54) Method for preventing and treating severe acute respiratory syndrome

(76) Inventor: Charles Hensley, Redondo Beach, CA (US)

Correspondence Address: TOD R NISSLE, PO BOX 55630, PHOENIX, AZ, 85078, US

(21) Appl. No.: 10/751,590

(22) Filed: Jan. 5, 2004

(51) Int. Cl. A61K 31/353; A61K 31/7048

(52) U.S. Cl. 514/456; 514/27

(57) ABSTRACT

A composition and method for preventing and treating SARS utilizes theaflavin and its derivatives in combination with a carrier.

This invention relates to methods and compositions for treating and preventing disease.

More particularly, the invention relates to a method and composition for treating and preventing SARS (severe acute respiratory syndrome).

SARS is a respiratory illness. This illness was first reported Asia in 2003. One symptom of SARS is a fever greater than 100.4 degrees F. Other symptoms can include body aches, an overall feeling of discomfort, a headache, and mild respiratory symptoms. Other patients can develop a dry cough and have difficult breathing. SARS is considered a serious health threat because the mortality rate appears to be around ten percent.

SARS is believed to be caused by a previously unrecognized coronavirus, called SARS-associated coronavirus. Other infectious agents may also play a part in SARS infections.

The Center for Disease Control (CDC) in the United States has apparently tested over 10,000 compositions against SARS and only two of these compositions were effective, a licorice component and an interferon component. Both of these components, however, have significant undesirable side effects.

Accordingly, it would be highly desirable to provide an improved method and composition for treating and preventing SARS.

Therefore, it is a principal object of the invention to provide an improved method and composition for preventing and treating SARS.

I have discovered a composition and method for treating and/or preventing severe acute respiratory syndrome in an individual.

The method comprises administering a composition including a carrier and an anti-coronaviral ingredient comprising theaflavin, theaflavin-3,3′-digal (TF-3), theaflavin-3-monogallate (TF-2), theaflavin-3 gallate, theaflavin-3′-gallate, and/or thearubigin. The carrier can be a liquid or solid or combination thereof, however a liquid carrier comprised of water and/or alcohol is presently preferred. The concentration of the anti-coronaviral ingredient can be in the range of 0.1% by weight to 90% by weight. The composition can be administered any desired number of times, but is presently preferably administered at least once a day for at least three days. If desired, the theaflavin (i.e., theaflavin, theaflavin-3,3′-digal (TF-3), theaflavin-3-monogallate (TF-2), theaflavin-3 gallate, theaflavin-3′-gallate) or thearubigin molecule can be modified by attaching different molecules to the theaflavin, by removing a portion(s) of the theaflavin molecule, or by removing a portion(s) of the theaflavin molecule and incorporating a different structure for the removed portion of the theaflavin molecule. The quantity of theaflavin molecules administered to a patient during a single treatment can vary as desired. However, concentrated theaflavin is presently preferred. A treatment program for a patient can comprise a single treatment or can comprise a plurality of treatments.

### EXAMPLE 1

A composition containing 99.9% by weight water (as a carrier) and containing 0.1% by weight of the anti-coronavirus component theaflavin is prepared by admixing water and theaflavin at room temperature.

### EXAMPLE 2

A composition containing 99.9% by weight water (as a carrier) and containing 0.1% by weight of the anti-coronavirus component theaflavin-3,3′-digal is prepared by admixing water and theaflavin-3,3′-digal at room temperature.

### EXAMPLE 3

A composition containing 99.9% by weight water (as a carrier) and containing 0.1% by weight of the anti-coronavirus component theaflavin-3-monogallate is prepared by admixing water and theaflavin-3-monogallate at room temperature.

### EXAMPLE 4

A composition containing 99.9% by weight water (as a carrier) and containing 0.1% by weight of the anti-coronavirus component theraflavin is prepared by admixing water and theraflavin-3 gallate at room temperature.

### EXAMPLE 5

A composition containing 99.9% by weight water (as a carrier) and 0.1% by weight of the anti-coronavirus component theaflavin-3′-gallate is prepared by admixing water and theaflavin-3′-gallate at room temperature.

### EXAMPLE 6

A composition containing 99.9% by weight water (as a carrier) and 0.1% by weight of the anti-coronavirus component thearubigin is prepared by admixing water and thearubigin at room temperature.

### EXAMPLE 7

Example 1 is repeated, except ethanol is utilized as a carrier in place of water.

### EXAMPLE 8

Example 2 is repeated, except ethanol is utilized as a carrier in place of water.

### EXAMPLE 9

Example 3 is repeated, except ethanol is utilized as a carrier in place of water.

### EXAMPLE 10

Example 4 is repeated, except ethanol is utilized as a carrier in place of water.

### EXAMPLE 11

Example 5 is repeated, except ethanol is utilized as a carrier in place of water.

### EXAMPLE 12

Example 6 is repeated, except ethanol is utilized as a carrier in place of water.

### EXAMPLE 13

Examples 1 to 12 are repeated, except that the concentration of the anti-coronavirus component is 5% instead of 0.1%.

### EXAMPLE 14

Examples 1 to 12 are repeated, except that the concentration of the anti-coronavirus component is 10% instead of 0.1%.

### EXAMPLE 15

Examples 1 to 12 are repeated, except that the concentration of the anti-coronavirus component is 50% instead of 0.1%.

### EXAMPLE 16

Examples 1 to 12 are repeated, except that the concentration of the anti-coronavirus components is 90% instead of 0.1%.

### EXAMPLE 17

Sixteen healthy adults are selected and are exposed to and directly contacted with the SARS virus such that it is highly likely each individual will develop symptoms associated with SARS. The adults are quarantined to prevent spread of the disease. Eight of the adults comprise the control group. Members of the control group that eat normal meals three times a day, exercise once a day for an hour, and read, watch television or play games. The control group does not receive any antibiotics or other compositions to treat or prevent the onset of SARS. The remaining eight adults comprise the test group. Members of the test group, like the control group, also eat normal meals three times a day, exercise once a day for an hour, and, read, watch television, or play games. In addition, the test group receives once a day two tablespoons of the composition of Example 1. During the next ten days after being exposed to the SARS virus, seven of the adults in the control group develop SARS symptoms including a fever greater than 100.4 degrees F., headache, body aches, and an overall feeling of discomfort. Two of the adults in the control group develop a dry cough and have labored breathing. None of the adults in the test group develop any SARS symptoms.

### EXAMPLE 18

Example 17 is repeated except that the composition of Example 2 is utilized in place of the composition of Example 1. Similar results are obtained.

### EXAMPLE 19

Example 17 is repeated except that the composition of Example 3 is utilized in place of the composition of Example 1. Similar results are obtained.

### EXAMPLE 20

Example 17 is repeated except that the composition of Example 4 is utilized in place of the composition of Example 1. Similar results are obtained.

### EXAMPLE 21

Example 17 is repeated except that the composition of Example 5 is utilized in place of the composition of Example 1. Similar results are obtained.

### EXAMPLE 22

Example 17 is repeated except that the composition of Example 6 is utilized in place of the composition of Example 1. Similar results are obtained.

### EXAMPLE 23

Example 17 is repeated except that the composition of Example 7 is utilized in placed of the composition of Example 1. Similar results are obtained.

### EXAMPLE 24

Example 17 is repeated except that the composition of Example 8 is utilized in place of the composition of Example 1. Similar results are obtained.

### EXAMPLE 25

Example 17 is repeated except that the composition utilized includes 95% by weight water and 5% by weight of theaflavin. Similar results are obtained.

### EXAMPLE 26

Example 17 is repeated except that the composition utilized includes 50% by weight water and 50% by weight of theaflavin. Similar results are obtained.

### EXAMPLE 27

Examples 17 to 26 are repeated except that the adults in the test group are given once a day ten tablespoons of the composition that includes the anti-coronavirus component. Similar results are obtained.

### EXAMPLE 28

Examples 17 to 26 are repeated except that the adults in the test group are given once a day one tablespoon of the composition that includes the anti-coronavirus component. Similar results are obtained.

The anti-coronavirus compositions of the invention can also be used to treat and prevents SARS virus induced immunopathology (including by not limited to inflammation cause by cytokines); to treat and prevent SARS infections in animals; and, as an agent to kill SARS virus on surfaces.

1. A method for treating and/or preventing severe acute respiratory syndrome in an individual comprising the step of administering to the individual a composition comprising theaflavin and a carrier.

2. A method for treating and/or preventing severe acute respiratory syndrome in an individual comprising the step of administering to the individual a composition comprising theaflavin-3,3′-digal (TF-3) and a carrier.

3. A method for treating and/or preventing severe acute respiratory syndrome in an individual comprising the step of administering to the individual a composition comprising theaflavin-3-monogallate (TF-2) and a carrier.

4. A method for treating and/or preventing severe acute respiratory syndrome in an individual comprising the step of administering to the individual a composition comprising theaflavin-3 gallate and a carrier.

5. A method for treating and/or preventing severe acute respiratory syndrome in an individual comprising the step of administering to the individual a composition comprising theaflavin-3′-gallate and a carrier.

6. A method for treating and/or preventing severe acute respiratory syndrome in an individual comprising the step of administering to the individual a composition comprising thearubigin and a carrier.
